(19) United States
(12) Patent Application Publication
(10) Pub. No.: US 2010/0100114 A1
(43) Pub. Date: Apr. 22, 2010

(54) Inflatable tissue elevator and expander

(76) Inventor: J. Lee Berger, Franklin Lakes, NJ (US)

Correspondence Address: JOHN S. HALE;GIPPLE & HALE, 6665-A OLD DOMINION DRIVE, MCLEAN, VA, 22101, US

(21) Appl. No.: 12/289,075

(22) Filed: Oct. 20, 2008

(51) Int. Cl. A61M 29/00

(52) U.S. Cl. 606/191

(57) ABSTRACT

A surgical instrument including a fixed or removable handle and a tubular member having a lumen extending therethrough with an apertures at its proximal end and a blunt dissecting distal tip. An access window in a distal portion of the instrument is formed through the wall of the tubular member and in communication with the lumen. A balloon catheter is permanently mounted in the lumen whereby inflation of the balloon causes it to expand through the access window.

### RELATED APPLICATIONS

None

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention relates to a cannulated tubular surgical tissue elevator and expander with a pre-mounted balloon catheter. The device may be used as a conventional manual elevator for orthopaedic or general surgery, and is particularly suited for use in accessing the carpal tunnel region during carpal tunnel release procedures.

2. Background of the Invention

The use of a cannula, sheath, or director to guide or ease insertion of surgical instruments or to facilitate access to a surgical field is well known. With particular reference to carpal tunnel syndrome, these devices are commonly used to facilitate the introduction of a balloon catheter that is serially inflated and allowed to deflate in the carpal canal during surgery to alleviate entrapment of the nerve.

Carpal tunnel syndrome is a common painful condition of the hand characterized by a decrease in median nerve sensibility with paresthesias of the fingers. It is the most common nerve entrapment syndrome of the upper extremity and results from compression of the median nerve in the carpal tunnel and its symptoms include nocturnal pain, clumsiness, and weakness of grasp or pinch.

The carpal canal is the area in the wrist and palm of the hand formed by the U-shaped cluster of bones of the carpus that forms the rigid hard floor and the two sides of the tunnel. Within the confines of this space are the median nerve, and the extrinsic flexor tendons of the thumb and fingers with their surrounding synovial membranes of the radial and ulna brusae. The roof of the tunnel is formed by the transverse carpal ligament on the palmar surface of the carpal bones. The ligament is attached medially to the pisiform and the hamulus of the hamate, and laterally to the tuberosity of the scaphoid and the medial part of the palmar surface and ridge of the trapezium. The proximal border of the ligament is partially merged to the distal border of the palmar carpal ligament, a more superficial structure separated from the transverse carpal ligament by the ulnar artery and nerve. The transverse carpal ligament is attached to the palmar aponeurosis, which lies superficial, and contributes crossed oblique fibers to the deep surface of the aponeurosis.

Any condition that diminishes the size of the carpal tunnel can precipatate the carpal tunnel syndrome. Repetitive wrist and hand motions can cause thickening and hypertrophy of the transverse carpal ligament, proliferation of synovium from normal wear and tear of daily activities can cause compression of the median nerve within the closed confines of the carpal tunnel. Furthermore, systemic conditions such as pregnancy, obesity, diabetes mellitus, thyroid dysfunction, or chronic renal failure can initiate the syndrome. Generally, the syndrome occurs most often in patients between the ages of 30 and 60 years and is five times more frequent in women than in men.

Failure to correct the condition in a timely manner ultimately results in irreversible muscle damage, as indicated by denervation potentials.

Historically, carpal tunnel syndrome has been treated nonsurgically by splinting of the affected hand and wrist, oral antiinflammatory medication, and local steroid injection. Where nonsurgical methods are unsuccessful, surgical intervention is necessary.

Open surgical decompression of the carpal tunnel by division of the transverse carpal ligament was first described in 1930 by Learmonth. Open procedures generally entail a curved longitudinal incision to the ulnar side and parallel to the thenar crease. Another open procedure angles the incision towards the ulnar aspect of the wrist helps to avoid cutting the palmar sensory cutaneous branch of the median nerve. After dissection of the skin and subcutaneous tissue, the transverse carpal ligament is identified and divided along its ulnar border to avoid and to prevent injury to the median nerve or its recurrent branch.

Open surgical decompression of the median nerve is not without risks and complications. Long term analysis of patients who have undergone surgical treatment for carpal tunnel syndrome have shown a failure rate as high as 19% (Kulick, Michael, I., D.D.S., M.D., et al.; Long-term analysis of patients having surgical treatment of carpal tunnel syndrome; Jour of Hand Surg; 11A (1):59-66. 1986). Thirty percent of all patients in one study were rated with poor or fair result at an average of 5.5 years follow-up “57% of the patients in the study complained of return of some preoperative symptoms, most commonly pain, beginning an average of 2 years after surgery.” (Nancollas, Michael P.; Symptoms may return after carpal tunnel surgery; JAMA,; Vol 265, No 15, p 1922, Apr. 17, 1991.) Complications include continued pain and or numbness, hypersensitive scar, loss of grip strength, joint stiffness, adherence of flexor tendons, neuroma, injury to the median nerve or its motor branch or digital nerve injury, damage to the palmar cutaneous nerve, vascular injury, palmar hematoma, infection, and possible reflex sympathetic dystrophy.

A retrospective analysis of 40 cases of reoperation for carpal tunnel syndrome found adhesions and fibrosis around the median nerve in the carpal canal in 36 cases. (Wadstroem, J., Nigst, H.,; Reoperation for carpal tunnel syndrome: a retrospective analysis of 40 cases; Ann. Chir. Main; 5:54-58, 986.)

Hand weakness with loss of grip strength can also be a problem. Carpal tunnel release produces an average widening of the transverse carpal arch of 2.7 mm. There is a direct relation-ship between widening of the carpal canal and decreased grip strength. The average decrease in grip strength is 12%. (Gartsman, Gary, M., Kovach, John, C., et al.; Carpal arch alteration after carpal tunnel release; J. Hand Surg. (AM.) 11-A:372-374, May, 1986.) Computerized tomographic (CT) studies of the carpal tunnel after transsection of the transverse carpal ligament have shown that if the flexor retinaculum is not intact,-the flexor tendons will bowstring palmarly when the wrist and fingers are flexed together, causing weakness of grip. Some authors have recommended the use of a firm postoperative compression dressing immobilizing the wrist in slight dorsal extension for 3 weeks to prevent prolapse of the flexor tendons. (Jessurun, W, Hillen, B, et al.; Carpal tunnel release: Postoperative care: Handchirurgie 20:3940, January, 1988.)

Because of the concern over widening of the carpal arch with subsequent decrease in grip strength, following standard carpal tunnel release, Jakab and associates devised a technique employing reconstruction of the transverse carpal ligament. (Jakab, Emery, Ganos, Doreen, et al.; Carpal tunnel release: Postoperative care: Handchirurgie 20:3940, January, 1988.) These authors learned that by reconstructing the transverse carpal ligament, the transverse carpal arch was stabilized affording protection to the median nerve and preventing potential bow-stringing of the flexor tendons. By repairing the transverse carpal ligament the normal relationship of the carpal canal and its contents was restored and maintained.

Pre and post operative CT scanning of the wrist in carpal tunnel syndrome has been per-formed and has shown that postoperative decompression results in the regeneration of a much more flexible ligament, which allows the contents of the tunnel, to expand anteriorly. The divided transverse carpal ligament heals in a stretched or arched position allowing more room for the median nerve and flexor tendons. (Chaise, F., Roger, B.,; Pre-and post-operative CT scanning of the wrist in carpal tunnel syndrome; Rev Chir. Orthop.; 72:297-302' 1986.) In recent years endoscopic techniques have been utilized to incise and divide the trans-verse carpal ligament. Attempts have been made to decrease the size of the surgical incision with the benefit of decreasing postoperative complications because hand strength returns more quickly and the hand is less tender.

Refinements of endoscopic release of the carpal ligament are evolving but complications with this procedure have been reported The complications include neuropraxia of the median and or ulnar nerve, digital nerve laceration, laceration of palmar vessels, laceration of flexor tendons, neuropraxia or laceration of the palmar cutaneous branch of the median nerve, palmar hematoma, adhesions of tendons, perineural fibrosis, bowstringing of flexor tendons with loss of grip strength, incomplete transsection of the carpal ligament with recurrence of symptoms and hypertrophic scar.

U.S. Pat. No. 5,179,963 issued Jan. 19, 1993 to the present inventor solves these aforementioned complications and employs a balloon catheter device with a separate nerve protector inserter and pressure gauge monitor. The balloon catheter is inserted percutaneously and is utilized to dilate and expand the transverse carpal ligament, through serial applications of fluid pressure while it is moved along the carpal tunnel, thereby increasing the diameter of the carpal tunnel, relieving compression of the median nerve and alleviating the symptoms of carpal tunnel syndrome. Percutaneous dilatation of the transverse carpal ligament increases the spatial diameter of the carpal tunnel, thereby relieving pressure on the median nerve in the hand and wrist without the surgical and treatment problems previously discussed.

U.S. Pat. No. 5,545,136 issued Aug. 13, 1996, also issued to the present inventor, discloses a surgical instrument used in the treatment of carpal tunnel syndrome having a rigid tubular member with lumen through which a balloon carpal tunnel plasty procedure may be undertaken. The balloon expands through an aperture in the radial wall of the tubular member and the tubular member includes a rigid, solid rounded tip to ease insertion of the apparatus into the carpal tunnel. Other instruments are then used in conjunction with the instrument to perform the surgery. U.S. Pat. No. 6,706,069 issued Mar. 16, 2004, also to the present inventor, is directed toward a grooved director with a built in balloon which is inflated by a pump to a predetermined pressure to expand the walls of a collapsed vertebra. The device is inserted into the body of the compressed vertebra and the grooved director is positioned and aimed in a direction under the compressed superior end plate of the vertebral body. The balloon inside of the grooved director is inflated and the force and direction of balloon inflation restores the height of the fractured vertebrae. The balloon is deflated and the grooved director is circumferentially rotated while intermittently inflating and deflating the balloon to create a symmetrical space within the center of the vertebral body. The balloon is deflated and the grooved director device with balloon is removed leaving a rebuilt vertebra which may be filled with a biocompatible material. U.S. Pat. No. 4,655,214 issued Apr. 7, 1987 shows a soft inflatable sheath having a closed rounded distal tip that is inserted through a catheter and inflated adjacent the distal tip of the catheter prior to intubation. The proximal end of the sheath is sealed to maintain it in an expanded condition when the catheter is being intubated. Following intubation the cylindrical sheath is deflated and withdrawn. U.S. Pat. No. 4,645,491 issued Feb. 24, 1987 shows a catheter placement apparatus used in inserting a catheter to a preferred depth. The device comprises a surgical needle provided with a thin-walled transparent polytetrafluoroethylene tube which is heat shrunk over the stem portion of the needle to form a longitudinal window allowing a catheter inserted in the needle to be viewed. The catheter has a colored patch of the same length as the window and a series of spaced circular bands of differing colors allowing the position of the catheter to be accurately located by lining the colored patch with the window and advancing the catheter until at least one band appears in the window. The color and distance of the band nearest to the surface of the patient's skin are used to determine the position of the catheter. The surgical needle is withdrawn by sliding it along and off the catheter. U.S. Pat. No. 2,164,926 issued Jul. 4, 1939 shows a catheter stylet with an eye or aperture positioned on an opposite lateral wall behind the tip. U.S. Pat. No. 3,537,452 issued Nov. 3, 1970 shows a needle guard and beveled cutter for use with intravenous catheterization units. The device has a tubular body with a flat base and a longitudinally slotted top. The diameter of the tube is greater than the diameter of the needle contained therein. U.S. Pat. No. 3,592,193 issued Jul. 13, 1971 shows a removable needle guide used with a flexible catheter tube in withdrawing or introducing fluids relative to a body. The hollow tubular needle guide has a sharpened needle portion provided at its proximal end for puncturing the skin, tissues and veins of the body where the needle is inserted. At its distal end, the guide has winged handles which provide controlled insertion and removal from the body with subsequent attachment from a flexible catheter tube. U.S. Pat. No. 5,011,478 issued Apr. 30, 1991 shows an introducer set including a sheath and dilator formed with a smooth external shape. The distal end of the sheath is embedded in the dilator and formed in angle oblique to the longitudinal access of the introducer set. U.S. Pat. No. 3,559,643 issued Feb. 2, 1971 shows a catheter placement unit for insertion of a catheter into a body lumen through an incised opening in the lumen wall. The unit includes a longitudinally slit sheath having a catheter therein and an advancer connected to one end of the catheter, initially in axial alignment with the sheath to close the end of the sheath.

There is presently a need, however, for an orthopedic and general surgical manual instrument that combines various features of multiple instruments. With regard to the carpal tunnel release procedures described above, an instrument similar to known elevator devices is needed to extend the reach of the surgeon into the confines of the operating field and facilitate elevation, retraction and/or manipulation of the tissues therein. Particularly regarding the surgical intervention described supra, the instrument of the present invention is uniquely combined with a balloon catheter permanently mounted within its hollow interior wherein its balloon may be deployed from a protected window in the distal portion of the instrument.

### SUMMARY OF THE INVENTION

The present invention is a multi-functional surgical tool intended for use as a conventional manual elevator for orthopaedic or general surgery having a balloon permanently mounted within a generally rigid, rod-like tubular member adjacent its distal end that may be inflated or deflated as desired by the user. Prior to deploying the balloon, the device is used to dissect, elevate, and retract tissues within the surgical field and its tubular structure partially encloses the balloon catheter within to protect it and facilitate its positioning prior to use. It is ideally suited for hand and wrist surgery, particularly with regard to endoscopic, balloon assisted carpal tunnel release. Additional surgical uses will be immediately obvious to those skilled in the art and include use in a variety of elbow, shoulder, hip, knee, ankle, and foot procedures.

In an alternative embodiment, the generally hemispherical closed distal tip of the expander and elevator described above is optically clear and refractive, and the instrument is sized to accommodate an arthroscope or endoscope. The aforementioned scope may then be inserted prior to inflation of the balloon whereby the visual field viewed through the closed distal tip is modified by the optical properties of the tip. The interior (i.e., facing the lumen of the present invention) or exterior surfaces of the tip may be ground with refractive indeces that, for example, permit wide angle viewing through the distal tip. Alternatively, the tip may be ground to provide a highly directed, magnified view, or the tip may simply be non-refractive and serve to protect the tip of an arthroscope or endoscope inserted therein. The tip of the scope may also be retracted slightly to permit viewing through the access window, in this instance with the tip of the scope being protected by the flaccid material of the pre-mounted balloon catheter.

In yet another alternative embodiment, the arthroscope or endoscope in the embodiment described above may incorporate inflation means, for example gas or fluid delivery mechanisms generally known to those in the field. The scope seals the inflation stem of the balloon catheter such that the balloon may then be pressurized while the arthroscope or endoscope is inserted.

In still another alternative embodiment of the present invention, its handle is removable.

It is an object of this invention to provide a multi-functional surgical instrument.

It is another object of this invention to provide a combination elevator, retractor, and balloon catheter for orthopedic and general surgery.

It is still another object of this invention to provide a multi-functional surgical instrument in multiple sizes to accommodate various surgical procedures and instrumentation.

It is an object of this invention to provide an inexpensive to manufacture, biologically inert, and autoclavable surgical instrument.

It is still yet another object of this invention to provide a protective barrier for arthroscopes and endoscopes.

It is an object of this invention to selectively modify the visual field of an arthroscope or endoscope as desired by the user.

The invention will be better understood and objects other than those set forth above will become apparent when consideration is given to the following detailed description thereof Such description makes reference to the annexed drawings wherein

### BRIEF DESCRIPTION OF THE DRAWINGS

FIG. 1 is a perspective view of the multi-functional inflatable tissue elevator and expander with balloon expanded;

FIG. 2 is a side elevation view of the invention depicted in FIG. 1 with its balloon expanded;

FIG. 3 is a side elevation view of the invention depicted in FIG. 2 with a flaccid balloon;

FIG. 4 is a side elevation view of the invention depicted in FIG. 2 with a portion of the invention broken away;

FIG. 5 is a side elevation view of the invention depicted in FIG. 3, with a tubular cover protecting its balloon and the distal portion shown in phantom;

FIG. 6 is an enlarged partial detailed elevation of the invention depicted in FIG. 5;

FIG. 7 is a front end elevation view of the invention depicted in FIG. 6; and,

FIG. 8 is a perspective view of an alternative embodiment of the present invention including a removable handle.

These and other objects, advantages, and novel features of the present invention will become apparent when considered with the teachings contained in the detailed disclosure along with the accompanying drawings.

### DESCRIPTION OF THE INVENTION

While the invention is described in connection with certain preferred embodiments, it is not intended that the present invention be so limited. On the contrary, it is intended to cover all alternatives, modifications, and equivalent arrangements as may be included within the spirit and scope of the invention as defined by the appended claims.

The invention is a cannulated, stepped and generally tubular instrument open at its proximal end. An access window is formed adjacent the distal end of the device and a balloon catheter is permanently mounted in an open bevel-ended section of the device whereby inflation of the balloon catheter causes it to expand through the access window. The device includes a blunt, closed distal tip useful for dissection of surrounding tissue during emplacement of the apparatus. A flattened, ergonomically shaped handle with a throughgoing bore is mounted at the proximal end over the tube proximal end to facilitate precise control of the device while in use. The handle is semi-circular in cross section with a flat top defining a finger recess. In an alternative embodiment, the handle is removable and one-half of one inch of the surface of the proximal end of the generally tubular portion of the device is knurled, thereby ensuring precise control is maintained whether the handle is used or detached.

The device can be manufactured from stainless steel, cobalt chrome, titanium, nitinol, other metals, polymers, ceramics, composites or other biocompatible materials, and can be manufactured in various sizes and dimensions.

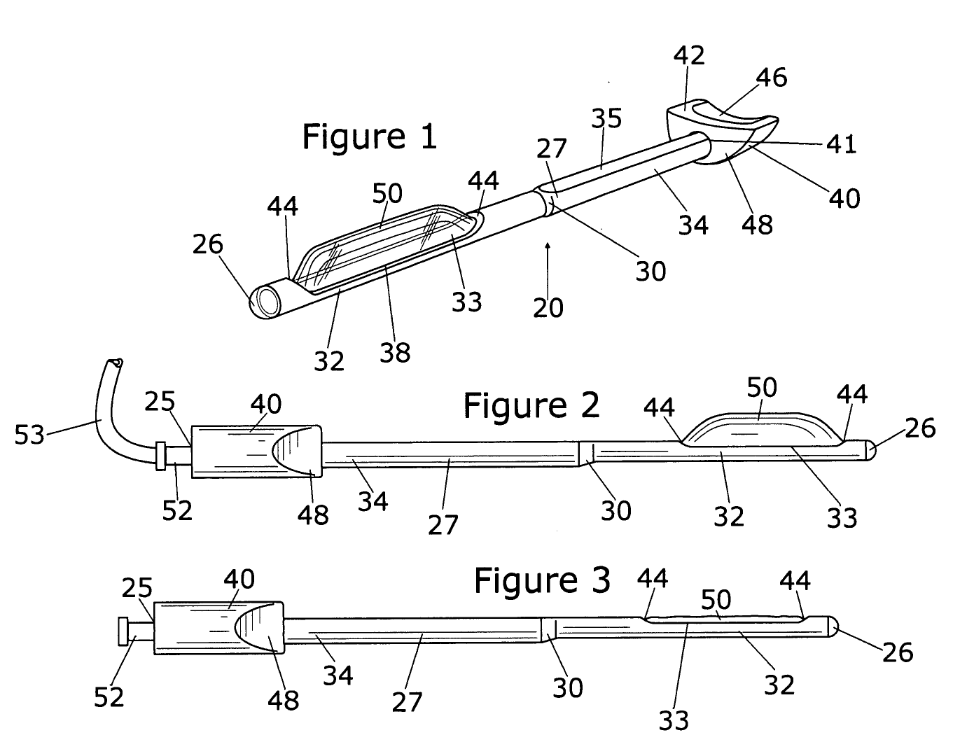

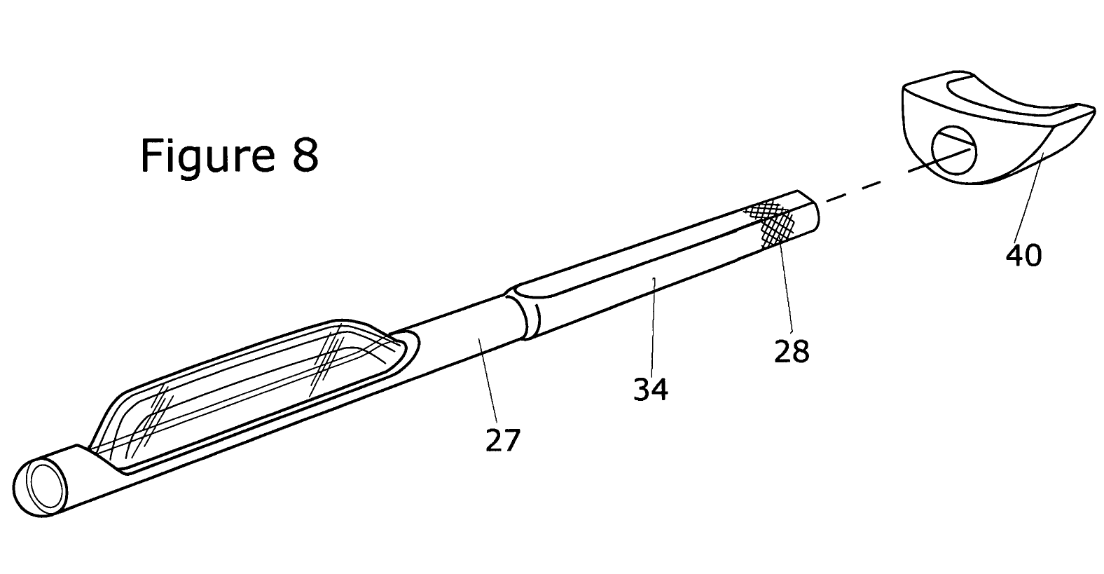

A preferred embodiment and best mode of the invention is shown in FIGS. 1-8. FIG. 8 shows an embodiment with a removable handle. The multi-functional, tissue expander and elevator 20 is constructed from surgical steel or polymer material forming its tubular member 27, with an ergonomically shaped handle 40 mounted at its proximal end. The tubular member 27 is cannulated, having a throughgoing, generally cylindrical lumen 24 extending throughout the length of the proximal section and on both ends of the distal section, with a proximal aperture 25 in communication with the lumen 24. A generally blunt, hemispherical closed tip 26 is mounted at the distal end of tubular member 27. The balloon catheter 50 of the present invention is fixedly secured to the interior surface of the closed tip 26 and the access window 33, thereby permanently mounting said catheter 50 within the lumen 24 of the device. Tubular member 27 is stepped at 30 to provide a thinner diameter distal tubular section 32 while providing strength near its handle 40 via proximal tube section 34. The proximal tube section 34 of the tubular member 27 proximal to step 30 defines a planar top surface 35 extending the length of the proximal section and functions to securely fix the handle 40 about the tubular member 27 and provide anti-roll characteristics to the member 27. The tubular member 27 is cut so that a portion of its wall is removed adjacent its distal end to form an access window 33 having beveled ends 44. The access window 33 is defined by radiused proximal and distal ends 44 and generally parallel side ends 38 of the distal tubular section 32.

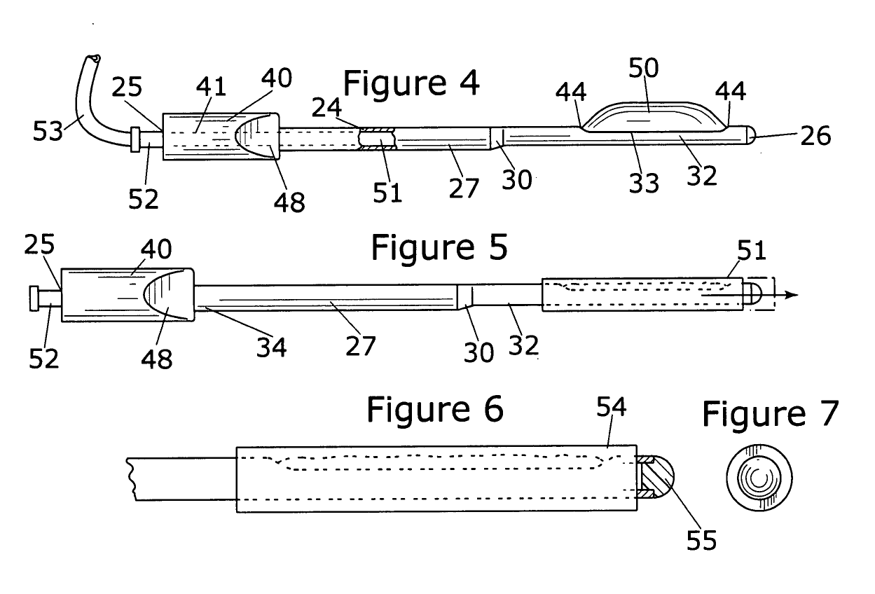

The handle 40 of the device is formed from a polymer material and is ergonomically shaped to facilitate handling of the instrument 20. In a preferred embodiment, the handle 40 is generally planar on its top surface 42 and defines a recessed concave surface 46 which accommodates the surgeon's thumb to ensure positive control of the grasped device. As seen most clearly in FIG. 4, a throughgoing lumen 41 extends through the handle and is axially aligned with and in communication with the proximal aperture 25 of the tubular member 27. The handle 40 has a semi-circular cross section and a round convex front surface 48. The handle 40 is permanently affixed to the tubular member 27 via an adhesive or sonic welding. If desired both the handle and tube can be formed from a single piece of material such as stainless steel or rigid medically approved plastic material, as for example polyethylene or polypropylene. The planar portion 35 of the tubular element 27 results in an eccentrically shaped cross section such that the corresponding handle lumen 41 ensures positive engagement of the handle 40 to the proximal end of the tubular element. The cross sections of the proximal end of the tubular element 27 and the handle lumen 41 ensure that when mated, the handle 40 and tubular element 27 are fixed and cannot rotate relative to one another.

FIG. 4 clearly depicts the balloon catheter 50 permanently mounted in the lumen 24 of the device, including an inflation stem 51 interconnecting the balloon 50 with an inflation fitting 52 mounted at the proximal end of tubular member 27 and adjacent handle 40. In FIG. 3, the balloon catheter 50 is depicted in a flaccid state. The inflation fitting 52 is a common luer lock style fitting, but any fitting suitable for sealably connecting the balloon catheter 50 to its pressurization source may be used. Such fittings include, but are not limited to, twistlock connectors, threaded connectors, and friction fittings.

In FIGS. 5 and 6, an optional tubular protective cover 54 is depicted. The protective cover 54 is dimensioned to closely match the diameter of the distal tubular section 32 and is frictionally mounted thereto. It protects the material of the balloon catheter prior to operational use and is generally removed at the commencement of surgery. Clearly depicted in FIG. 6 is the visualization lens 55 used in lieu of the closed tip 26 in an alternative embodiment. As shown here, the lens 55 is generally optically transparent wherein the expensive optics of an arthroscope or endoscope inserted into this otherwise inexpensive device will be protected while in use. Additionally, the optical properties of the generally convex cross section of the lens 55 results in a wider field of view regarding objects viewed therethrough. It is contemplated that the visualization lens 55 may be ground with additional refractive indeces as desired, for example, the lens 55 may be ground to provide a magnified view of tissues viewed therethrough. In yet another embodiment, the arthroscope or endoscope used in conjunction with the aforedescribed embodiment can include a mechanism to pressurize the balloon catheter 50 of the device 20. Hence, the expander and elevator may be used without first withdrawing the arthroscope or endoscope prior to inflation of the balloon catheter 50.

FIG. 8 depicts the surgical tissue expander and elevator 20 of the present invention with a removable handle 40. The handle 40 is frictionally mounted to the proximal tube section 34 of tubular member 27 and may be detached as desired. The proximal tube section 34 of the tubular member 27 includes knurling 28 to ensure positive handling of the tubular element 27 when the handle 40 is not attached and to increase friction and positive attachment of the handle 40. When handle 40 is not attached, the planar portion 35 of the tubular element 27 minimizes the opportunity for the tubular element 27 to roll off the work surface.

In a preferred use, an incision is first cut through the skin and subcutaneous tissue by sharp dissection. The expander and elevator 20 is then used to elevate or retract tissues whereby its blunt tip 26 ensures dissection of any necessary tissues away from the surgical field without damage. Once emplaced, a pressurization source 53 is attached to inflation fitting 52 thereby permitting inflation of balloon 50 and expansion of tissues at the surgical site as desired. The device may be used for these and additional functions known to those skilled in the art, with and without additional support.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A surgical tissue expander and elevator comprising:
an elongated substantially rigid tubular member dimensioned to be inserted through tissue and defining a longitudinal axis, said tubular member having a proximal end portion and a distal end portion with a distal-most closed tip, said tubular member defining a lumen extending therethrough with a balloon catheter permanently secured therein, said balloon catheter having an inflation stem extending through said lumen and terminating adjacent said tubular member proximal end portion, a distal-most portion of said lumen terminating at said distal-most closed tip, said tubular member including an access window in an outer wall portion thereof in communication with said lumen, said window being disposed intermediate said proximal and distal end portions of said tubular member such that a distal-most portion of said lumen is disposed beyond said opening, said access window dimensioned to permit expansion of a balloon of said balloon catheter therethrough upon inflation of the balloon to permit the expanded balloon to engage and dilate tissue, and a handle defining gripping means mounted at said proximal end.

2. A surgical tissue expander and elevator as claimed in claim 1 wherein said distal-most portion of said lumen is dimensioned to receive and accommodate a distal end of said balloon catheter and further wherein distal axial movement of the catheter relative to said tubular member is limited by engagement of the catheter distal end with said distal-most closed tip of said tubular member

3. A surgical tissue expander and elevator as claimed in claim 1 wherein said distal-most closed tip is a visualization lens and said balloon catheter inflation stem is sized to accept an arthroscope or endoscope for visualization of body structures.

4. A surgical tissue expander and elevator as claimed in claim 3 further including an arthroscope or endoscope sealingly mountable in said balloon catheter inflation stem, said arthroscope or endoscope including balloon catheter inflation and deflation means therein.

5. A surgical tissue expander and elevator as claimed in claim 3 wherein said access window is sized to permit comprehensive visualization of the surgical site via an arthroscope or endoscope inserted into said balloon catheter inflation stem.

6. A surgical tissue expander and elevator as claimed in claim 3 wherein said closed tip comprising said visualization lens is a blunt closed tip and said visualization lens is refractive.

7. A surgical tissue expander and elevator as claimed in claim 1 wherein surface edges of said outer wall portion defining and surrounding said single opening are chamfered to avoid penetration of said balloon upon expansion thereof through said access window.

8. A surgical tissue expander and elevator as claimed in claim 1 wherein said handle defines a central throughgoing bore adapted to fit over the proximal end of said tubular member and defines a planar top surface with at least one finger recess.

9. A surgical tissue expander and elevator as claimed in claim 8 wherein an inflation fitting in communication with said inflation stem is sealably mounted to a proximal end of said central throughgoing bore of said handle, said fitting being selected from a group consisting of a luer lock, a twistlock, a threaded fitting, and a friction fitting.

10. A surgical tissue expander and elevator as claimed in claim 1 wherein said handle is removable from said tubular member and said gripping means is a finger recess.

11. A surgical tissue expander and elevator as claimed in claim 10 wherein said removable handle is a friction fit at said proximal end with the cross-section of at least the said proximal end of said tubular member being non-circular.

12. A surgical tissue expander and elevator as claimed in claim 1 wherein said tubular member defines a distal section and a stepped proximal section, said proximal section defining a planar surface coincident with and generally parallel to the longitudinal axis of said tubular member.

13. A surgical tool comprising:
an elongated substantially cylindrical rigid tubular member, said tubular member having a blunt closed distal end, a proximal section defining a planar surface and a stepped distal section, said tubular member defining a lumen extending therethrough with a balloon catheter mounted therein, said balloon catheter being inflatable via an inflation stem extending through said lumen, said inflation stem terminating in an inflation fitting mounted adjacent a proximal end of said tubular member, said tubular member defining an access window in said distal section in communication with said lumen, said access window dimensioned to permit expansion of a balloon of said balloon catheter, and a handle mounted on said proximal end of said tubular member.

14. A surgical tool as claimed in claim 13 wherein said inflation stem of said balloon catheter is sized to accept a surgical tool taken from the group consisting of surgical probes, ultrasound devices, electrical devices, magnetic devices, and radiofrequency devices.

15. A surgical tool as claimed in claim 13 wherein said distal section has a visualization lens mounted at its distal end in communication with said lumen.

16. A surgical tool as claimed in claim 13 wherein said handle is substantially semi-circular in cross section and ergonomically shaped with a finger recess defined on a top planar face.

17. A surgical tool as claimed in claim 16 wherein said handle is a removable handle which is a friction fit on said tubular member proximal end and a proximal section of said tubular member is knurled.

18. A surgical tool as claimed in claim 13 wherein said tubular member includes a planar surface coincident with and generally parallel to the longitudinal axis of said tubular member.

19. A surgical tool as claimed in claim 13 wherein said access window is sized to permit comprehensive visualization of the surgical site via an endoscope or arthroscope inserted into a lumen of said balloon catheter.

20. A surgical tissue expander and elevator comprising:
an elongated substantially rigid tubular member defining a longitudinal axis, said tubular member having a distal blunt tip dimensioned to be inserted through tissue, said tubular member having a proximal section with a non-circular cross section and planar outer surface and a distal section with an open elongated access window bevelled at each end, said tubular member defining a lumen extending therethrough and defining an aperture at said proximal end in communication with said lumen and said access window, said tubular member having a removable ergonomically shaped handle mounted at said proximal end, and a balloon catheter permanently mounted in said lumen, an inflation stem of said balloon catheter extending through said lumen and terminating at an inflation fitting mounted adjacent said proximal end.
